(19) United States
(12) Patent Application Publication
(10) Pub. No.: US 2011/0206695 A1
(43) Pub. Date: Aug. 25, 2011

(54) ANTIBODIES FOR DIAGNOSIS AND THERAPEUTIC TREATMENT OF PROSTATE CANCER

(75) Inventors: Manu Kohli, Dunn Loring, VA (US); Michael Goldblatt, McLean, VA (US); Michael Kinch, Laytonsville, MD (US)

(73) Assignee: FUNCTIONAL GENETICS, INC., Gaithersburg, MD (US)

(21) Appl. No.: 13/013,002

(22) Filed: Jan. 25, 2011

Related U.S. Application Data
(60) Provisional application No. 61/297,887, filed on Jan. 25, 2010.

(51) Int. Cl. A61K 39/395; C12Q 1/70; A61P 35/00

(52) U.S. Cl. 424/172.1; 435/5

(57) ABSTRACT

XMRV appears to be related to both prostate cancer if it infects a male germ cell and chronic fatigue syndrome in both sexes. (If the virus does not infect a germ cell). Prostate cancer cells exhibit TSG101 on the surface only upon infection with a virus like XMRV. Antibodies to TSG101 can be effective diagnostics to identify individuals with a predisposition to prostate. They can also be used in place of current diagnostics to confirm the presence of prostate cancer. TSG101 antibodies, when administered in vivo, exhibit the ability to reduce tumor size, suppress metastatic transformation and extend survival.

### PRIORITY DATA AND INCORPORATION BY REFERENCE

This application claims benefit of priority to U.S. Provisional Patent Application No. 61/297,887 filed Jan. 25, 2010 which is incorporated by reference in its entirety.

### BACKGROUND OF THE INVENTION

1. Field of the Invention

This invention pertains to detection of Xenotropic Murine Leukemia-Virus Related virus, or XMRV, as well as detection and possible treatment of disease states associated with that virus, including metastatic prostate cancer and Chronic Fatigue Syndrome, or CFS.

2. Related Art

This invention relates to the detection of the presence of TSG101 protein on the surface of cells of mammalian hosts suspected of being infected with XMRV. In particular, XMRV has recently been found to be associated with malignant prostate cancer cells. Fan, PNAS, Vol. 101, 5, 1449-11450 (2007). TSG101 is a protein ordinary found in the cytoplasm of healthy mammalian cells, and is conserved in mammals. TSG101 is instrumental as a member of the family of ESCRT protein in directing proteinacious material within cell for storage and destruction. In the event of infection by many enveloped viruses, it appears that the normal function of TSG101 is “hijacked” by the infecting virus. In the event of infection by a variety of viruses, TSG101 is found on the cell surface of the infected cell. This phenomenon, and the ability to bind to the TSG101 and thereby inhibit viral infectivity, is reported in U.S. patent application Ser. No. 11/940,714, the entirety of which is incorporated by reference.

The binding of TSG101 on the cell surface, as well as other ESCRT proteins like Nedd4 is also discussed in U.S. patent application Ser. No. 11/939,122 filed Nov. 30, 2007, also incorporated herein by reference. Interference with the activity of TSG101 in a virally infected cell poses so many potential anti-viral treatments that small molecule binding, which would not be limited to cell surface phenomena, also provides therapeutic treatment, as reported in U.S. patent application Ser. No. 12/261,603 filed Oct. 30, 2008. All of these cases are directed to the identification and treatment of disease states associated with viral infection itself, such as influenza, HIV/AIDS, RSV and related viral diseases.

The recent identification of XMRV as highly associated with aggressive, metastatic prostate cancer presents an opportunity to diagnose and treat this most common of deadly male cancers, if a way to identify the infection of prostate cancer cells by XMRV can be found. Researchers have postulated that perhaps a genetic defect in RNase L, an effector in the interferon induced innate response may provide a favorable opening for the virus. In these individuals, a single aa mutation, R462Q leads to reduced enzyme activity which may be all that is necessary to allow the retrovirus to successfully infect the cell. If the virus manages to infect a germ cell, any oncogene (typically captured) may be integrated into the DNA of the host cell, and passed on as stably inherited elements. This may in fact be the causal link between prostate cancer and XNRV that many are looking for. It is, in any event, clear that XMRV infection is found in a high percentage of prostate tumor cells, particularly aggressive metastatic ones.

At the same time, researchers have found that sixty-seven percent (67%) or more of humans suffering from Chronic Fatigue Syndrome (CFS) are infected with XMRV. While the causal relationship is not clear, in fact, the degree of association is so high that XMRV is implicated by many as instrumental in the course of this chronic disease. The probability that the retrovirus is a causative agent is reinforced by the fact that a large majority of CFS patients first exhibit CFS symptoms following a period of illness in which they exhibit flu-like symptoms. XMRV, like HIV, is a retrovirus that has a long resident life, and thus could be causing the initial flu-like infection, followed by a period of residency in which the CFS symptoms are manifested. Typically CFS persists for life. A large proportion of these patients exhibit pain and immune problems and CFS is highly correlated with Fibromyalgia, which may be simply an extreme reaction to the viral infection.

Current methods of diagnosis of prostate cancer are limited. The two diagnostics widely available are a digital rectal exam, where a medical professional palpates the prostate through the rectum to try and detect hard lumps or anomalies. The other is an assay for “prostate specific antigen” or PSA. Elevated PSA may indicate prostate cancer. Neither diagnostic is complete. Many men with high PSA levels do not have prostate cancer. Many types of prostate cancer, and many aggressive but early stage cancers, are not detectable by digital exam. In either event, a questionable result is followed by biopsy and tissue analysis and MRI or similar imaging.

There are few effective cures for viral infection, and fewer vaccines. Therapeutics tend to be targeted at the specific virus, which will mutate to escape the effectiveness of the therapeutic, and, like HIV, can remain at rest in the body for many years. As of the filing date of this application, there was neither treatment for, nor vaccine to prevent, XMRV infection. When the sequelae of the infection manifest, such as aggressive metastatic prostate cancer, or CFS, treatment is largely limited to tumor attack and patient support.

### SUMMARY OF THE INVENTION

Applicants have now demonstrated that cells, in particular, prostate cells, infected with XMRV, can be detected by antibody binding to TSG101 on the surface of the cells, where uninfected cells show no binding (by staining) by the same antibodies. Antibodies, both polyclonal and monoclonal, to TSG101 are widely available. This presents a new, powerful method to detect XMRV infection. Male patients testing positive for XMRV infection should be considered in a higher risk category for development of aggressive prostate cancer, and appropriate diagnostics, behavior modification and therapy initiated.

Cells of mammals, including humans, can also be assayed for the genetic defect in RNase L. Individuals not displaying symptoms of prostate cancer, CFS or XMRV infection may be candidates for vaccination to induce the expression of anti-TSG101 antibodies. An effective circulating titer of such antibodies has been shown to inhibit viral proliferation—the virus cannot escape the infected cell with budding, so that the infection does not spread. Passive protection through the administration of human or humanized anti-TSG101 antibodies may also be effective.

### BRIEF DESCRIPTION OF THE DRAWINGS

The accompanying drawings, which are incorporated herein and constitute part of this Specification, illustrate exemplary embodiments of the invention, and, together with the general description given above and the detailed description given below, serve to explain the features of the invention. The drawings depict experiments involving TSG101 surface staining on 22Rv1 prostate cancer cells which are chronically infected with actively budding xenotropic murine leukemia virus-related virus (XMRV).

FIG. 1 is the histogram of staining of the surface of infected cells with rabbit IgG—a control.

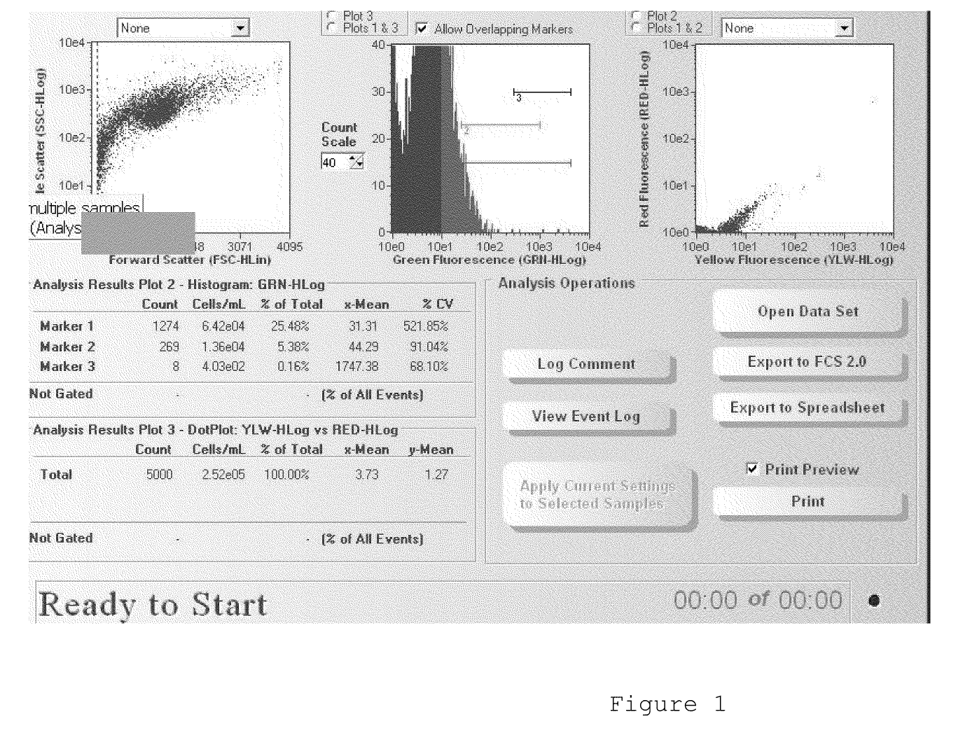

FIG. 2 is the histogram of staining of the surface of infected cells with a polyclonal antibody positive for TSG101—antibody 1299.

FIG. 3 is the histogram reflecting surface staining of XMRV infected cells with a human IgG control.

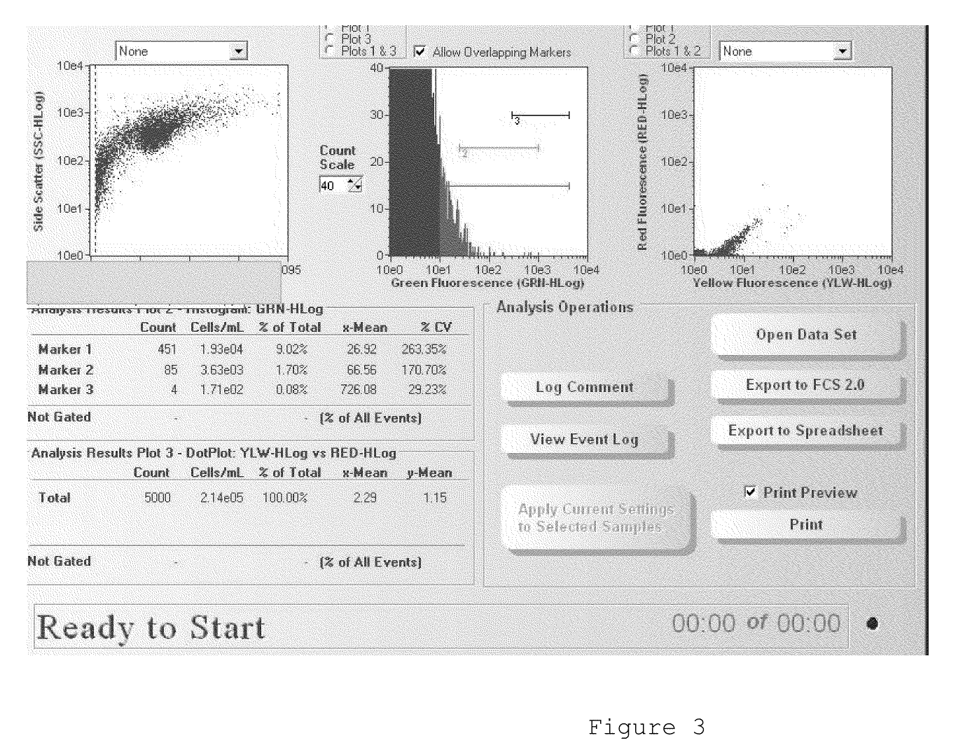

FIG. 4 is the histogram showing staining of XMRV infected cells with a monoclonal anti-TSG101 antibody which is the subject of a deposit made under Budapest Treaty conditions, antibody CB8-2.

FIG. 5 is a graph demonstrating the presence of TSG101 on the surface of prostate cells infected with XMRV, as opposed to non-infected cells, using an anti-TSG101 antibody through fluorescent activated cell sorting (FACS).

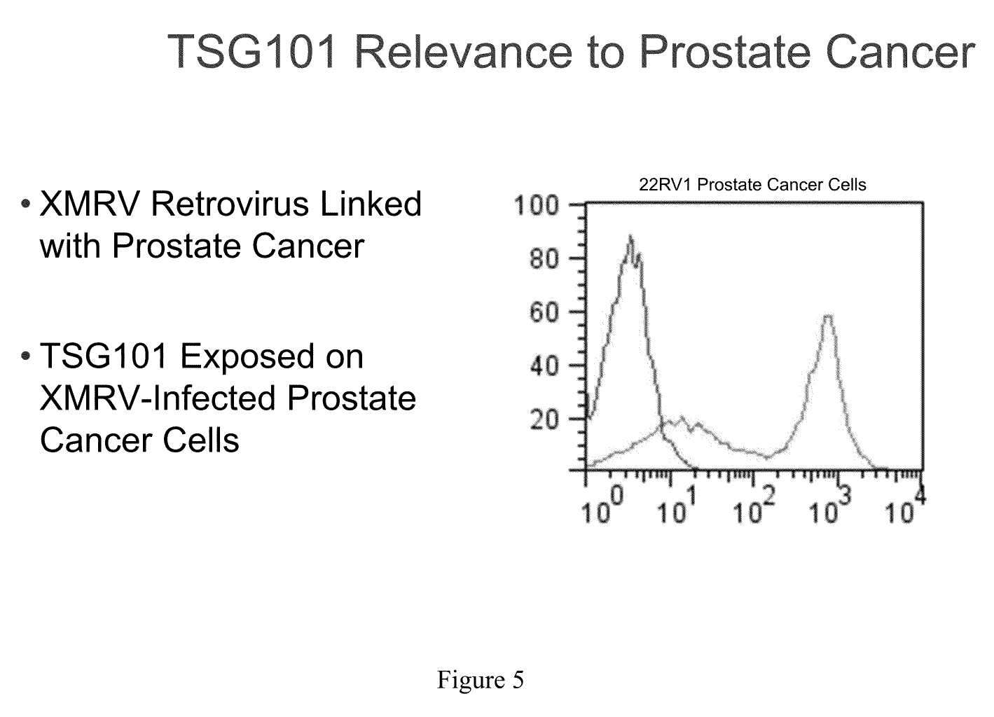

FIG. 6 reflects staining observed in five (5) different specimens of prostate cancer tissue, when stained with the anti-TSG101 antibody currently moving forward in clinical trials—CB-8 also known as FGI-101-1A6, deposited at the ATCC under Budapest Treaty conditions PTA-9611.

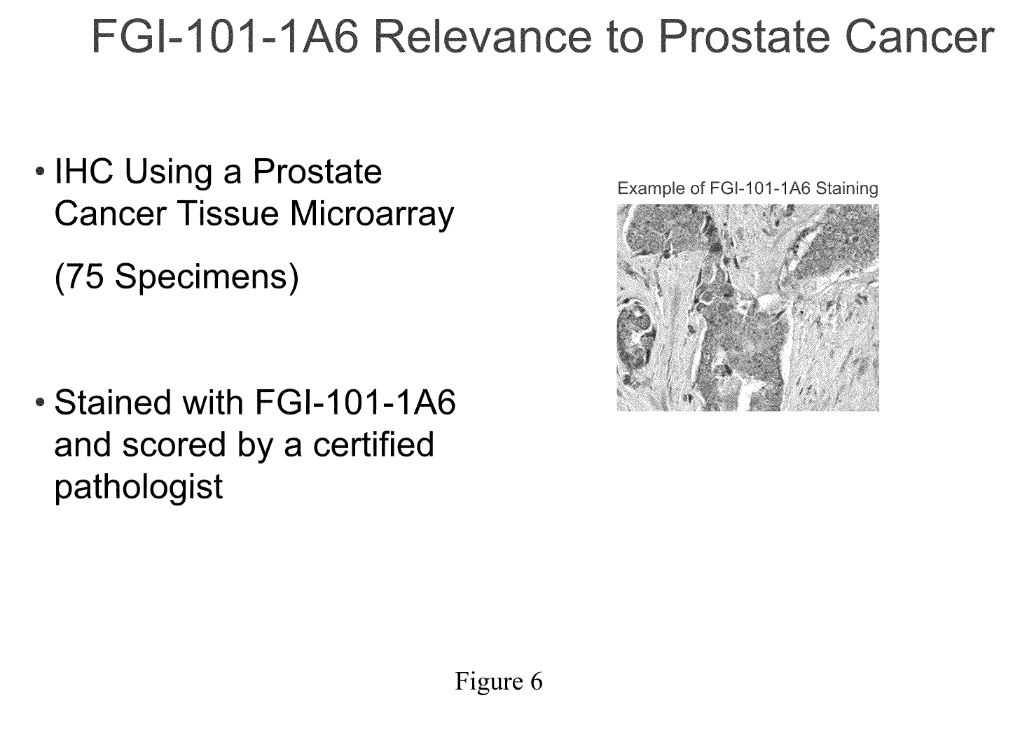

FIG. 7 is a schematic reflecting the results of the screening discussed in FIG. 6 above, showing a high correlation between staining and malignant prostate cancer specimens, suggesting that TSG101 is reflected on the surfaces of the most aggressive XMRV infected cells.

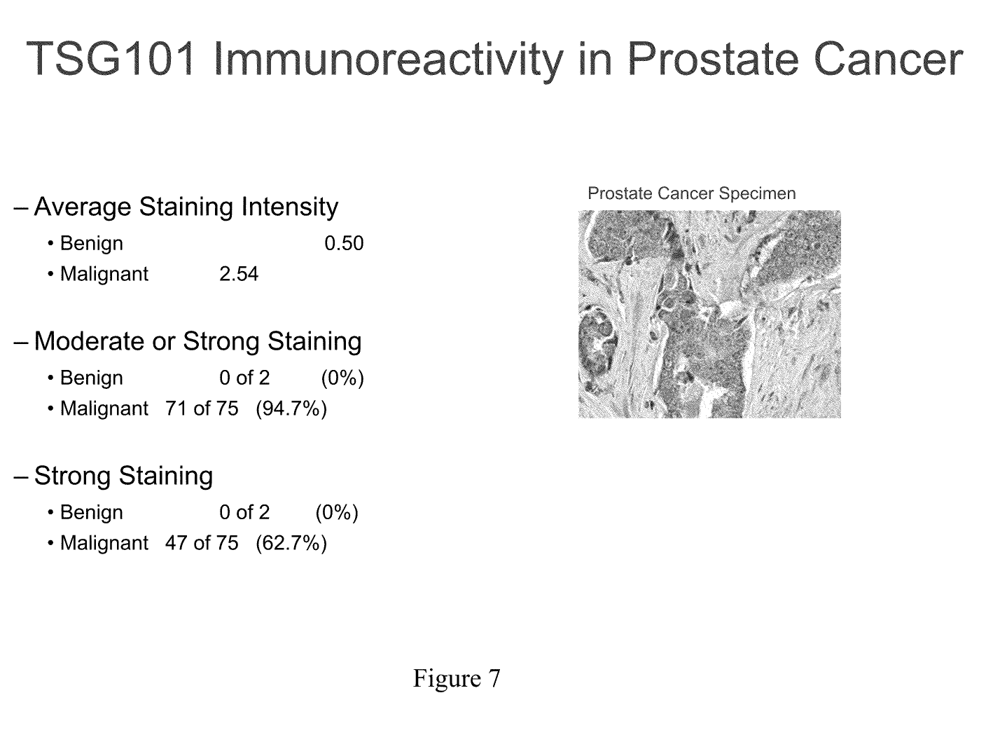

FIG. 8 is a schematic describing in vivo trials of TSG101 antibodies as a therapeutic in the treatment of XMRV-related prostate cancer.

FIG. 9 is a graphic reflection of in vivo studies showing the administration of a low circulating titer of TSG101 successfully reduced tumor growth and at least extended survival of mice challenged with subcutaneously implanted CWR22-Rv1 prostate cancer cells subcutaneously.

### DETAILED DESCRIPTION OF THE INVENTION

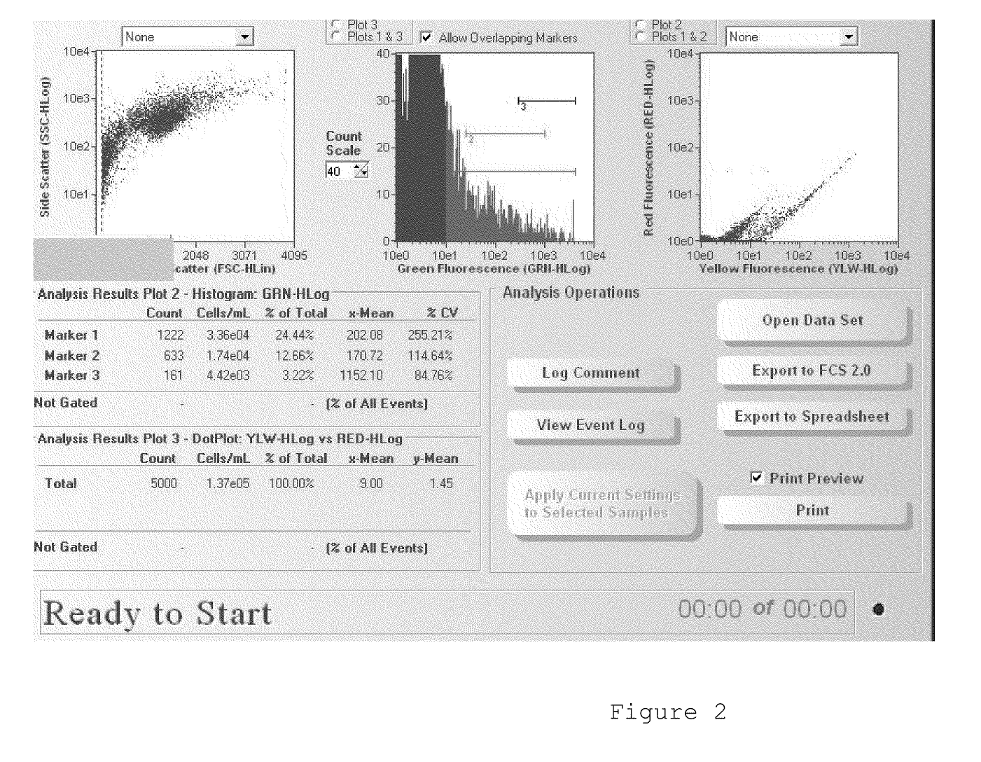

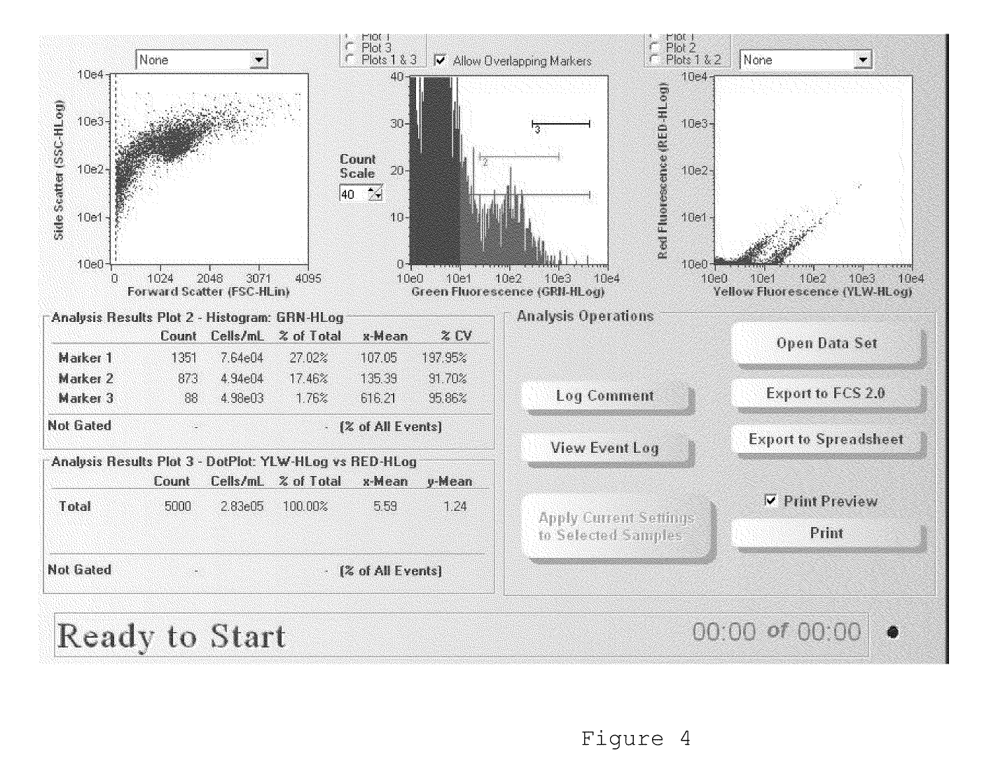

The four histograms shown clearly demonstrate that viral infection of prostate cancer cells alters the normal biology of the cell in a variety of ways, including delivering TSG101 to the surface of the cell, where it may be captured by an antibody specific for this protein. The cell line used in these assays, 22Rv1 prostate cancer cells, are merely representative of prostate cell lines and prostate cancer cell lines. The “right shift” shown in FIGS. 2 and 4 make it clear that in cells infected with XMRV, TSG101 can be found on the cell surface. As related in U.S. patent application Ser. No. 11/939,122, and U.S. patent application Ser. No. 11/940,714, both incorporated herein-by-reference, antibodies specific for TSG101 do not bind to the surface of the cell in the absence of viral infection. Thus, it appears that XMRV infection, like infection by other retroviruses like HIV, and other lethal viruses, like influenza and ebola, causes TSG101 to be manifested on the surface of the cell.

This binding phenomenon offers a variety of opportunities for diagnosis and possibly treatment. As a diagnostic, virtually any antibody that binds TSG101 selectively can be used—many are available commercially. The polyclonal antibody used to generate the data in FIG. 2 is a polyclonal antibody generated by immunizing a rabbit host with a TSG101 fragment comprising the UEV domain of TSG101. Such purified TSG101 peptides are disclosed and claimed in U.S. Pat. No. 5,807,995. Antibodies that selectively bind to TSG101 are disclosed and claimed in U.S. Pat. No. 6,835,816. Standard staining procedures for prostate cells should permit identification of individuals who stand an elevated chance of developing prostate cancer. Perhaps more importantly, early prostate cancer patients should be similarly assayed. Infection with XMRV in the prostate cancer cells is strongly indicative of a likelihood of aggressive, metastatic potential for these cancers, suggesting more pro-active treatment and monitoring than might otherwise be employed.

It is early in the course of research on XMRV infection, and it is not totally clear, at this time, whether or not it is in fact the XMRV infection itself that causes the prostate cancer tumors to acquire their metastatic character. A platform for the identification of therapeutic targets, RHGP, can itself be used to ascertain what factors or changes in a genome may cause a cancer tumor to acquire metastatic character, as disclosed in U.S. patent application Ser. No. 12/652,877 filed Jan. 6, 2010, incorporated by reference herein. Work is already underway to couple this powerful target identifier with the recognition that XMRV may be implicated in metastatic prostate cancer development.

As demonstrated in pending U.S. patent application Ser. No. 11/940,714, administration of certain anti-TSG101 antibodies that are effective in preventing virion release, and infected cell lysis, in vivo, have been shown to provide protection against viral challenge, in both whole cell and animal studies. Currently, studies to see if the same protection is conferred on primates are being pursued. A prostate cancer patient, in particular, is a likely beneficiary of administration of these antibodies, including antibody CB8-2, expressed by the cells of ATCC Deposit PTA-9611, and antibody 48A-4 (also referred as 15-2) expressed by the cells of ATCC Deposit PTA 10135. Both deposits were made pursuant to Budapest Treaty conditions. Suppression of possible XMRV infection, in these individuals, should serve to suppress the tendency of the prostate cancer cells to become metastatic, greatly improving morbidity and mortality projections.

For the same reasons, CFS sufferers should benefit from administration of anti-TSG101 antibodies. Protective anti-TSG101 antibodies might be administered to patients experiencing flu or flu-like symptoms. XMRV infection leading to CFS appears to gain a foothold through this type of infection. While undoubtedly, some of those so treated may be suffering from influenza infection, as opposed to XMRV, as detailed in U.S. patent application Ser. No. 11/940,714, these antibodies are effective against influenza infection as well—demonstrating the “pan-viral nature of antibodies directed against proteins like TSG101 and Nedd4, implicated in the life cycle of a wide variety of viral invaders. Thus, over, as opposed to under, identification of potential XMRV infection and CFS candidates is preferred.

The effective vaccination of individuals with TSG101 peptides and proteins to induce a circulating TSG101 antibody titer that is refreshed on challenge to suppress viral infectivity, and thereby avoid or prevent wide spread viral infection, is also discussed in U.S. patent application Ser. No. 11/940,714. As the target, TSG101, is not present on the surface of cells in the absence of viral infection, the antibody titer necessary to suppress viral infectivity is well tolerated. As a particular class of individuals meriting vaccination against TSG101 as a target, those individuals exhibiting the single amino acid genetic defect R462Q in RNase L that is associated with prostate cancer in males may be identified. The same “opening” provided the virus in prostate cells may allow the virus to infect and propagate in female mammals, including humans, and males, in non-germ cells, leading to CFS. Early screening to identify those members of the population with the R462Q mutation should be followed with TSG101 vaccination to provide protection against XMRV viral infectivity.

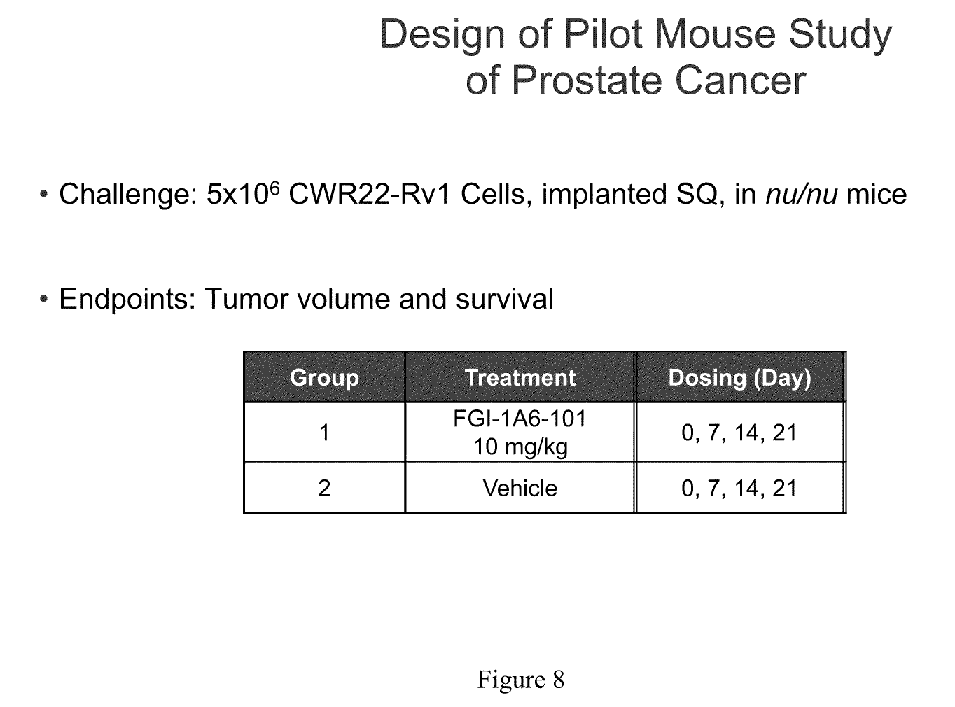

To further demonstrate the efficacy of treatment with TSG101 antibodies in controlling prostate cancer linked to XMRV infection, immunodeficient nu/nu nude mice were challenged with XMRV infected prostate cancer cells. Specifically, CWR11-Rv1 prostate cancer cells, shown to be XMRV infected, were subcutaneously injected into nu/nu mice. The prostate cancer cells spontaneously generate tumors in the mice which will grow, if not treated, and become lethal. This is a well-established challenge design for testing the efficacy of antibodies intended to control the growth and transformation of otherwise metastatic cancer. The design and parameters of the study are reflected in FIG. 8. As shown, the control for the study was the vehicle without any TSG101 antibody present.

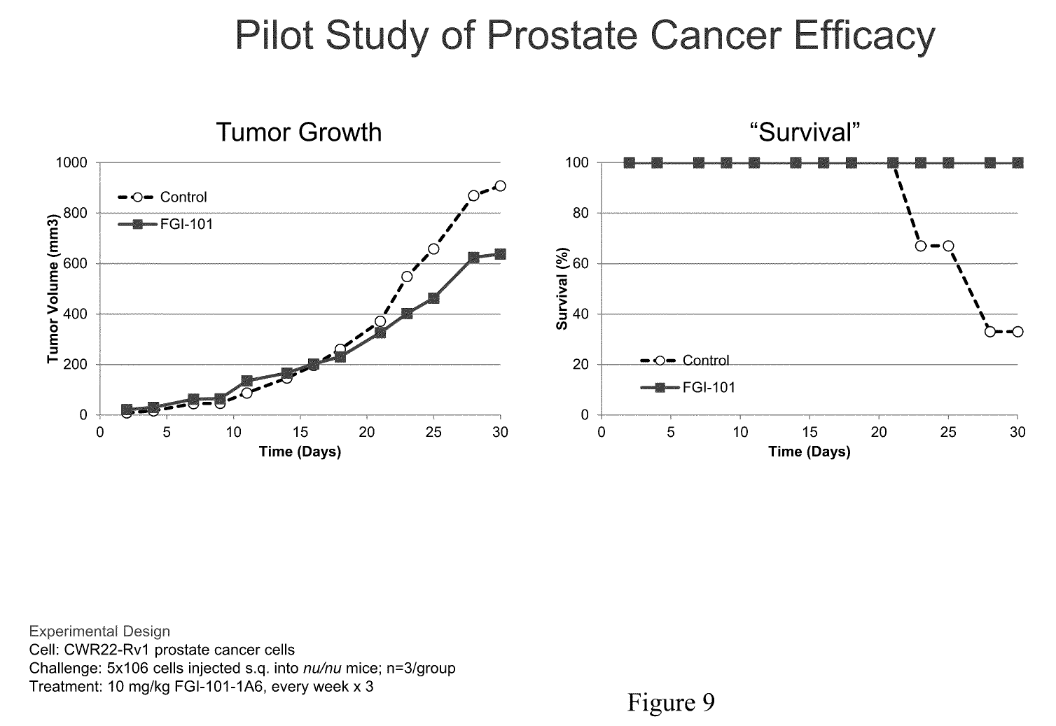

The results are reflected in FIG. 9. As can be seen, over a period of thirty (30) days, with a relatively minor dose of antibody administered on a weekly basis over a period of three (3) weeks, mammals receiving the antibody showed a significantly reduced level of tumor growth. Even more importantly, treated animals showed 1005 survival over the same period, where two-thirds of the test mammals did not. While further experiments are ongoing, as noted, TSG101 on the membrane surface of XMRV infected cells most highly correlates with malignant prostate cancer cells. It may be that not only does TSG101 antibody administration provide a protection against XMRV infection, but that protection in fact reduces or suppresses transformation of the cancer cells into aggressive growth and metastatic cancer.

As noted above, this invention contemplates both an easily performed diagnostic and screening test, but a treatment test. The diagnostic eliminates many of the suspect cases generated by digital examination or prostate specific antigen testing. Moreover, the screening procedure here is two-fold. Detection of XMRV infection suggests the individual positive for TSG101 may exhibit either prostate cancer or precancerous cells, and be a target for Chronic Fatigue Syndrome. Administration of TSG101 antibodies, or vaccination with an immunogenic TSG101 polypeptide, may in fact either suppress both prostate cancer formation and CFS, or at least reduce cancer formation and transformation. XMRV infection in female mammals, including humans, and non-germ cells of males, may be specifically related to CSF, and identify candidates for early treatment of, or suppression of, CFS and associated fibromyalgia. For these types of screenings, the antibodies may be polyclonal or monoclonal.

Importantly, XMRV infection tagged by anti-TSG101 antibodies in prostate cells appears to be associated with the most aggressive prostate cancers, including those likely to become metastatic. Accordingly, a different type of screening is provided when assessing prostate cancer patients. Such patients, and in particular early prostate cancer patients, should be screened for TSG101 present on the surface of the prostate cells. Such patients are likely to harbor more aggressive and pro-metastatic cancer types, and should be more aggressively treated, accordingly. Study after study demonstrates that even aggressive cancers can be controlled, if detected early enough.

The invention also provides for two (2) types of therapeutic intervention. Patients with a specific genetic defect, R462Q RNase L, correlate highly with XMRV infection and prostate cancer. The reduced enzyme activity associated with the genetic defect may well make it easier for this particular virus to infect the cell, in due course, giving rise to DFS and if the cell is a male germ cell, prostate cancer. Patients with the R462Q RNase L genetic alteration should of course be assayed for the presence of XMRV infection. Patients who do not show such infection, should then be vaccinated against infection, or treated with antibodies to provide a protective titer, of anti-TSG101 antibodies, that bind TSG101 on the surface of the cell targeted by XMRV, which appears to control infectivity and either prevent or limit cell infection by the virus.

Finally, those individuals found to be both suffering from prostate cancer or CFS and exhibiting XMRV infection may be benefitted by having TSG101 antibodies administered. In test mammals, this demonstrated a profound effect in both reducing tumor size and in extending survival. Since the binding of TSG101 on the surface of virally infected cells has been demonstrated effective in reducing the infectivity of influenza, HIV and Ebola, among others, and effective in extending survival of infected animals, it is no surprise that administration of similar or the same antibodies may suppress the consequences of infection by another virus, XMRV. Thus, prostate cancer patients may be either vaccinated with an immunogenic polypeptide that generates the expression of anti-TSG101 antibodies in the host in an effective circulating titer, or be provided with passive protection in the form of TSG101 antibodies such as those deposited at the ATCC in deposits PTA-9611 and PTA-10135, or similar antibodies demonstrated to be suitable to administration to mammals including humans, and having similar binding properties, including the ability to promote ADCC and or Fc mediated cell killing.

While the present invention has been disclosed with references to certain embodiments, numerous modification, alterations, and changes to the described embodiments are possible without departing from the sphere and character of the present invention. In particular, specific antibodies, cell lines, peptides and procedures are exemplary only. Alternatives, including dosing regimens and identification of appropriate dosage levels, are known to those of skill in the art, or could be readily determined by routine laboratory evaluation and testing.

While the present invention has been disclosed with references to certain embodiments, numerous modification, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of diagnosing a mammal to determine if the mammal is likely to develop prostate cancer, comprising:
contacting a prostate cell of said mammal with antibodies to TSG101, wherein binding of said antibodies to TSG101 on the surface of said cell is indicative of xenotropic murine leukemia virus (XMRV) infection of said cell; and
wherein mammals with prostate cells infected with XMRV are likely to develop prostate cancer.

2. The method of claim 1, wherein said antibodies are polyclonal.

3. The method of claim 1, wherein said antibodies are monoclonal.

4. A method of suppressing the development of XMRV-related prostate cancer in an mammal with a likelihood of developing XMRV related prostate cancer, comprising:
identifying mammalian individuals who exhibit a R462Q genetic mutation in RNase L, as individuals with a likelihood of developing XMRV-related prostate cancer; and
administering to said identified individuals a circulating titer of anti-TSG101 antibodies sufficient to bind TSG101 on a cell membrane of prostate cells of said individuals and thereby suppress XMRV infection in said cells.

5. A method of inhibiting XMRV-related prostate cancer in a male mammal, comprising providing to said male mammal an amount of anti-TSG101 antibody sufficient to bind TSG101 on a surface of a prostate cell of said male mammal.

6. The method of claim 5, wherein said method inhibits the formation of prostate cancer tumors in said male mammal.

7. The method of claim 5, wherein said administration of TSG101 antibodies inhibits the transformation of prostate cancer into metastatic cancer.

8. The method of claim 5, wherein said method extends the survival of said male mammal.

9. The method of claim 5, wherein said antibodies are monoclonal antibodies.

10. The method of claim 9, wherein said antibodies have the TSG101 binding properties of antibodies from ATCC deposit PTA-9611 or PTA-10135.

11. The method of claim 5, wherein said antibodies are provided by vaccinating said male mammal with an immunogen that generates the expression of TSG101 antibodies in said male mammal.

12. A method for treating XMRV-related chronic fatigue syndrome (CFS) in a mammal, comprising administering an amount of TSG101 antibodies that is sufficient to bind TSG101 on the surface of cells infected with XMRV.

13. The method of claim 12, wherein said method comprised administering TSG101 antibodies to said mammal in an amount that is sufficient to bind TSG101 on the surface of cells infected with XMRV.

14. The method of claim 12, wherein said antibodies are provided by vaccinating said mammal with an immunogen that generates the expression of TSG101 antibodies in said male mammal.

15. A method of treating a mammal, comprising administering to said mammal an amount of antibodies that bind specifically to TSG101 (TSG101 antibodies) sufficient to bind TSG101 on a surface of cells of said mammal infected with XMRV.
